(19) United States
(12) Patent Application Publication
(10) Pub. No.: US 2004/0047823 A1
(43) Pub. Date: Mar. 11, 2004

(54) Use of ergothioneine and/or its derivatives as an anti-pollution agent

(76) Inventors: Philippe Catroux, Nogent-sur-Marne (FR); Jose Cotovio, Danmartin-en-Goele (FR); Daniel Duche, Paris (FR)

Correspondence Address: D Douglas Price, Steptoe & Johnson, 1330 Connecticut Avenue NW, Washington, DC, 20036, US

(21) Appl. No.: 10/276,542

(22) PCT Filed: May 14, 2002
(86) PCT No.: PCT/FR01/01451

(51) Int. Cl. A61K 7/48

(52) U.S. Cl. 424/70.11

(57) ABSTRACT

The invention concerns the use of ergothioneine and/or its derivatives for topical application as anti-pollution agent, preferably as cosmetic anti-pollution agent, and a cosmetic treatment method using ergothioneine and/or its derivatives.

The present invention relates to the use in topical application of ergothioneine and/or its derivatives as an antipollution agent, and also to a cosmetic treatment process using ergothioneine and/or its derivatives.

Urban environments are regularly subjected to peaks of pollution. The atmospheric pollutants that are widely represented by the primary and secondary products of combustion represent a major source of environmental oxidative stress. Urban pollution is composed of various types of chemical products, xenobiotics and particles. Three major categories of pollutants can exert deleterious effects on the skin and the hair: gases, heavy metals and particles that are combustion residues, onto which are absorbed a large number of organic compounds.

It is the outermost tissues that are initially and directly exposed to environmental toxic agents. The skin is directly and frequently exposed to the prooxidizing environment. The environmental sources of oxidizing agents include oxygen, solar UV radiation and also, in polluted air, ozone, nitrogen oxides and sulfur oxides. The atmospheric pollutants represented by the primary and secondary products of domestic and industrial combustion such as monocyclic and polycyclic aromatic hydrocarbons are also a major source of oxidative stress. The skin is particularly sensitive to the action of oxidative stress and the outermost layer serves as a barrier against oxidative damage. In most circumstances, the oxidizing agent is likely to be neutralized after reaction with keratin materials, but the reaction products formed may be responsible for attacks on cells and tissues.

The stratum corneum, which is the skin's barrier, is the site of contact between the air and skin tissue. The lipid/protein two-phase structure is a crucial factor of this skin barrier function. These elements can react with oxidizing agents and be impaired, which will promote the phenomena of desquamation.

Ozone-induced lipid peroxidation can impair the skin in two ways:

1/The oxidation and degradation of the lipids of the stratum corneum can impair the barrier function of the stratum corneum. Disruption of the outer lipids and of the protein architecture appear to be triggering factors in many dermatoses (psoriasis, atopic dermatitis and irritant dermatitis).

2/The increased formation of lipid-oxidation products in the upper layers of the skin can trigger attacks in the adjacent layers of skin. The reaction of ozone (O₃) with unsaturated lipids involves addition reactions onto the double bonds. This process leads in a second stage to cleavage of the lipid chains and to the formation of aldehyde hydroperoxides and of hydrogen peroxide.

This involves a specific mechanism that is different from the lipoperoxidation mechanism conventionally described, which is mediated by a radical. The ozone-induced products of secondary or tertiary lipid oxidation, which are less reactive than ozone but have a longer lifetime, can propagate the effect of ozone. On account of their relative stability, the products of lipid oxidation and peroxidation, i.e. cholesterol oxides and aldehydes, have the potential to impair cells at remote sites not directly exposed to O₃.

A significant level of oxidative attack on the surface layers of the stratum can initiate localized subjacent inflammatory processes, leading to the recruitment of phagocytes, which, by generating oxidizing agents, will amplify the initial oxidative processes.

In urban pollution, concomitant exposure to O₃ and to UV A, B and/or C can cause synergistic oxidative stress.

Similarly, it may be thought that there is synergistic action between ozone and combustion-derived organic compounds.

Among the pollutants that can exert deleterious effects on keratin materials, toxic gases, such as ozone, carbon monoxide, nitrogen oxides or sulfur oxides, are one of the major constituents.

It has been found that these toxic gases, and more particularly ozone, promote the desquamation of keratin materials, make said keratin materials dirty and dull. Similarly, these gases are liable to cause cellular asphyxia in the epidermis.

Particularly, cellular asphyxia of keratin materials in the presence of ozone has been found. At high dose, this gas can also induce direct cytotoxicity, capable of resulting in cellular necrosis and alkylation of cellular nucleophiles, these mechanisms possibly being the cause of cutaneous sensitization or carcinogenesis.

Thus, there is a need for compositions that can prevent the harmful effects caused by these pollutants such as ozone, so as to protect keratin materials.

It has now been found, entirely surprisingly, that the use of ergothioneine and/or its derivatives makes it possible to protect keratin materials against the effects of pollutants.

Thus, the main subject of the invention is the use in topical application of ergothioneine and/or its derivatives, as an antipollution agent, and preferably as an antipollution cosmetic agent.

The expression “antipollution cosmetic agent” means an agent that protects the skin and keratin materials so as to prevent, attenuate and/or suppress the deleterious effects of toxic gases such as ozone.

Ergothioneine and/or its derivatives may especially correspond to those of formula (I):

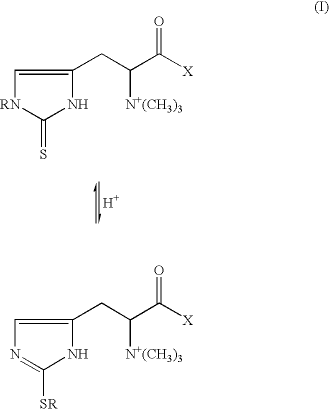

in which:

X represents a group —O—R′ or —NRR′;

R, R′ and R″ represent, simultaneously or independently of each other, a hydrogen atom, an alkyl group, an alkenyl group or an acyl group of C₁-C₁₈, or one of the salts of the compounds of formula (I).

The expression “C₁-C₁₈ alkyl, alkenyl or acyl group” means linear or branched alkyl, alkenyl or acyl groups containing from 1 to 18 carbon atoms, optionally substituted with a hydroxyl group (possibly being esterified itself), a halogen atom, a carboxylic group and its derivatives or an amine group and its derivatives, and possibly containing in the chain hetero atoms such as O or S.

Preferably, R, R′ and R″ denote, independently of each other, a hydrogen atom or an ethyl, acetyl or acyl group and X denotes NH₂.

Even more preferably, the compound of formula (I) is ergothioneine and especially L-(+)-ergothioneine, L-(−)-ergothioneine, D-(+)-ergothioneine or D-(−)-ergothioneine. The preferred compound according to the invention is L-(+)-ergothioneine.

Ergothioneine and/or its derivatives, as defined above, is (are) especially used to protect keratin materials against the effects of toxic gases and more particularly ozone.

In the context of the present invention, the expression “keratin material” especially means the skin, the scalp, the hair, the eyelashes, the eyebrows, the nails and mucous membranes.

Ergothioneine and/or its derivatives may also be used as an antipollution agent, preferably as an antipollution cosmetic agent, for improving cell respiration and/or for reducing desquamation and/or for preventing keratin materials from becoming dull or dirty.

A subject of the invention is also the use of ergothioneine and/or its derivatives in or for the preparation of an antipollution composition, which is preferably a cosmetic composition, for topical application.

It has also been found that these compositions have good stability properties, both in acidic medium and in basic medium. Finally, since ergothioneine has the advantage of being odorless, the compositions used in the invention will readily be accepted by consumers.

This composition may especially contain from 0.005% to 10% and preferably from 0.01% to 5% by weight of active material of ergothioneine and/or its derivatives relative to the total weight of the composition.

This composition may also contain at least one other antipollution compound. Said compound may be chosen especially from anthocyans and/or derivatives thereof, compounds containing a thioether, sulfoxide or sulfone function, heavy metal-chelating agents such as, for example, N,N′-dibenzylethylenediamine-N,N′-diacetic acid derivatives, antioxidants, ellagic acid or extracts of plants such as those of the Pontederiacea family.

The cosmetic compositions used according to the invention also comprise a cosmetically acceptable medium that consists more particularly of water and/or optionally of a cosmetically acceptable organic solvent.

The organic solvents may represent from 5% to 98% of the total weight of the composition. They may be chosen from the group consisting of hydrophilic organic solvents, lipophilic organic solvents and amphiphilic solvents, or mixtures thereof.

Among the hydrophilic organic solvents that may be mentioned, for example, are linear or branched lower monoalcohols containing from 1 to 8 carbon atoms, for instance ethanol, propanol, butanol, isopropanol and isobutanol, polyethylene glycols containing from 6 to 80 ethylene oxides, polyols such as propylene glycol, isoprene glycol, butylene glycol, glycerol, sorbitol, monoalkyl or dialkyl isosorbide, the alkyl groups of which contain from 1 to 5 carbon atoms, for instance dimethyl isosorbide, glycol ethers, for instance diethylene glycol monomethyl ether or monoethyl ether, and propylene glycol ethers, for instance dipropylene glycol methyl ether.

Lipophilic organic solvents that may be mentioned, for example, include fatty esters such as diisopropyl adipate, dioctyl adipate and alkyl benzoates.

Amphiphilic organic solvents that may be mentioned include polyols such as derivatives of propylene glycol (PPG), such as esters of polypropylene glycol and of fatty acids, derivatives of PPG and of fatty alcohols, for instance PPG-23 oleyl ether, and PPG-36 oleate.

In order for the compositions used in the invention to be more pleasant to use (softer to apply, more nourishing and more emollient), it is possible to add a fatty phase to the medium of these compositions.

The fatty phase preferably represents from 0% to 50% relative to the total weight of the composition.

This fatty phase may comprise one or more oils preferably chosen from the group consisting of:

volatile or nonvolatile, linear, branched or cyclic, organomodified or non-organomodified, water-soluble or liposoluble silicones,

mineral oils such as liquid paraffin and liquid petroleum jelly,

oils of animal origin such as perhydrosqualene,

oils of plant origin such as sweet almond oil, avocado oil, castor oil, olive oil, jojoba oil, sesame oil, groundnut oil, macadamia oil, grapeseed oil, rapeseed oil or coconut oil,

synthetic oils such as purcellin oil and isoparaffins,

fluoro oils and perfluoro oils,

fatty acid esters such as purcellin oil.

Said fatty phase may also comprise as fatty substances one or more fatty alcohols, fatty acids or waxes (paraffin wax, polyethylene wax, carnauba wax or beeswax).

In a known manner, the compositions used in the invention may also contain adjuvants that are common in cosmetics, chosen from the group consisting of standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, dyestuffs, bactericides and antidandruff agents.

The amounts of these various adjuvants are those conventionally used in the fields under consideration.

Needless to say, a person skilled in the art will take care to select the optional compound(s) to be added to the composition according to the invention, such that the advantageous properties intrinsically associated with the composition in accordance with the invention are not, or are not substantially, adversely affected by the envisioned addition.

The compositions used according to the invention may be in any presentation form normally used for topical application, especially in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase using spherules, these spherules possibly being polymer nanoparticles such as nanospheres and nanocapsules, or better still lipid vesicles of ionic and/or nonionic type.

The compositions used in the present invention may be more or less fluid and may have the appearance of a white or colored cream, an ointment, a milk, a lotion, a serum, a paste or a mousse.

They may optionally be applied to the skin in aerosol form.

They may also be applied in solid form, and for example in the form of a stick.

They may be used as care products and/or as makeup products.

The compositions according to the invention may have a pH of between 3 and 8 and preferably between 5 and 7.

Another subject of the invention consists of a cosmetic treatment process for protecting the body against the effects of pollution, which consists in applying to keratin material a cosmetically effective amount of ergothioneine and/or its derivatives as defined above.

Another cosmetic treatment process according to the invention, for protecting the body against the effects of pollution, consists in applying to keratin material a cosmetic composition according to the invention, as defined above.

The examples that follow are intended to illustrate the invention without, however, being limiting in nature.

### EXAMPLE I

### Demonstration of the Protective Effect of Ergothioneine

I. Protocol:

In a first stage, human keratinocytes obtained from plastic surgeries, inoculated beforehand on D-3 into a 48-well dish at a rate of 25 000 cells/cm², are pretreated with ergothioneine dissolved in the culture medium for 24 hours, at a final concentration of 0.5 mM.

A marker, 0,2,7-dichlorofluorescine diacetate, is then incorporated: after removing the ergothioneine solution and rinsing with PBS, the human keratinocytes are placed in contact for 30 minutes with a solution of 2,7-dichlorofluorescine diacetate (320 μM in phosphate-buffered saline PBS) at a rate of 500 μl per well, and are then rinsed again with PBS.

The cells are then exposed to ozone: after placing the cells in contact with a fresh solution of ergothioneine in PBS at a concentration of 1 mM, at a rate of 100 μl per well, the cells are exposed to ozone in an incubator (37° C., humid atmosphere) with an ozone concentration of 10 ppm.

The effects induced by the ozone are then measured by measuring the appearance of 2,7-dichloroflorescine (DCF: excitation at 485 nm and emission at 530 nm) after different exposure times: 0, 5, 10 and 20 minutes.

II. Results:

Four independent experiments were performed and each measurement was repeated six times.

The results are given in the table below:

Toxicity of ozone towards human keratinocytes in culture, in the absence or presence of ergothioneine at a concentration of 1 mM, as a function of the exposure time (n=4).

Fluorescence observed in the presence
of ergothioneine, expressed as % relative
to the unprotected controls for each time

| 5 min. of contact | 10 min. of contact | 20 min. of contact |
|---|---|---|
| % fluorescence | % fluorescence | % fluorescence |
| observed | observed | observed |
| ±SEM | ±SEM | ±SEM |
| 18.9 ± 2.4 | 25.9 ± 3.8 | 51.4 ± 10.2 |

The appearance of ozone-generated peroxides in the cell is reflected by an increase in the fluorescence. For each time, the fluorescence values of the unprotected controls are set at 100%. The results, in the presence of ergothioneine, are then expressed relative to this control value. A reduction in this value indicates a protective effect of the extract.

III. Conclusions:

In the presence of a high concentration of ozone, ergothioneine significantly reduces the effects induced by ozone. This protection is at a maximum from 5 minutes and 10 minutes of exposure onward (81.1% and 74.1% drop in induced stress). It is still significant after 20 minutes of exposure (48.6% drop in induced stress).

FORMULATION EXAMPLES

### Example 1

### According to the usual preparation techniques, the constituents below are mixed together to prepare an emulsion.

### Composition for Topical Application

| L-(+)-ergothioneine in which R = H, R′ = Ethyl, | 0.1 | g |
|---|---|---|
| R′′ = H and X = NH₂ | | |
| polyethylene glycol oxyethylenated with | 3 | g |
| 50 mol of ethylene oxide | | |
| monodiglyceryl stearate | 3 | g |
| liquid petroleum jelly | 24 | g |
| cetyl alcohol | 5 | g |
| water | qs | |
| | 100 | g |

### Example 2

n the same manner, an emulsion is prepared according to a standard technique, using the following compounds:

| ergothioneine in which R = H, R′ = Ethyl, | 0.1 | g |
|---|---|---|
| R′′ = H and X = OH | | |
| octyl palmitate | 10 | g |
| glyceryl isostearate | 4 | g |
| liquid petroleum jelly | 24 | g |
| vitamin E | 1 | g |
| glycerol | 3 | g |
| water | qs | |
| | 100 | g |

### Example 3

Starting with the constituents below, the following composition is formulated:

| L-(+)-ergothioneine in which R = H, | 0.02 | g |
|---|---|---|
| R′ = Ethyl, R′′ = methyl and X = NH₂ | | |
| jojoba oil | 13 | g |
| methyl isopropyl para-benzoxybenzoate | 0.05 | g |
| potassium sorbate | 0.3 | g |
| cyclopentadimethylsiloxane | 10 | g |
| stearyl alcohol | 1 | g |
| stearic acid | 4 | g |
| polyethylene glycol stearate | 3 | g |
| vitamin E | 1 | g |
| glycerol | 3 | g |
| water | qs | |
| | 100 | g |

### Example 4

Starting with the constituents below, the following composition is formulated:

| L-(+)-ergothioneine in which R = H, R′ = | 2 | g |
|---|---|---|
| Ethyl, R′′ = H and X = NH₂ | | |
| liquid paraffin | 15 | g |
| methyl isopropyl para-benzoxybenzoate | 0.05 | g |
| potassium sorbate | 0.3 | g |
| cyclopentadimethylsiloxane | 10 | g |
| sorbitol | 5 | g |
| ellagic acid | 0.1 | g |
| stearyl alcohol | 1 | g |
| stearic acid | 4 | g |
| polyethylene glycol stearate | 3 | g |
| vitamin E | 1 | g |
| glycerol | 3 | g |
| water | qs | |
| | 100 | g |

1. The use in topical application of ergothioneine and/or its derivatives as an antipollution agent, preferably as an antipollution cosmetic agent.

2. The use as claimed in claim 1, characterized in that ergothioneine and/or its derivatives correspond to formula (I): 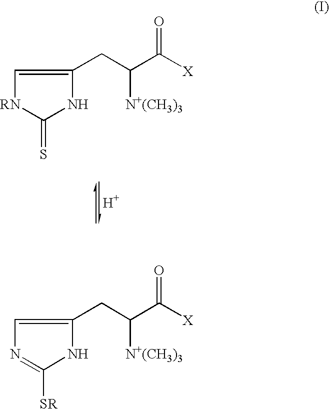
in which:
X represents a group —O—R′ or —NRR′;
R, R′ and R″ represent, simultaneously or independently of each other, a hydrogen atom, an alkyl group, an alkenyl group or an acyl group of C₁-C₁₈, or one of the salts of the compounds of formula (i), the linear or branched alkyl, alkenyl or acyl groups containing from 1 to 18 carbon atoms, optionally substituted with a hydroxyl group, possibly being esterified, a halogen atom, a carboxylic group and its derivatives or an amine group and its derivatives, and possibly containing in the chain hetero atoms such as O or S.

3. The use as claimed in claim 2, in which the groups R, R′ and R″ of the compound of formula (I) denote, simultaneously or independently of each other, a hydrogen atom or an ethyl, acetyl or acyl group and X denotes NH₂.

4. The use as claimed in any one of claims 1 to 3, in which the compound of formula (I) is ergothioneine.

5. The use as claimed in any one of claims 1 to 4, in which the compound of formula (I) is L-(+)-ergothioneine.

6. The use in topical application of ergothioneine and/or its derivatives as defined in any one of claims 1 to 5, as an antipollution agent, preferably as an antipollution cosmetic agent, for protecting keratin materials against the effects of toxic gases.

7. The use as claimed in claim 6, characterized in that ergothioneine and/or its derivatives as defined in any one of claims 1 to 5 make it possible to protect keratin materials against the effects of ozone.

8. The use in topical application of ergothioneine and/or its derivatives as defined in any one of claims 1 to 5, as an antipollution agent, preferably as an antipollution cosmetic agent, for improving cell respiration and/or for reducing desquamation and/or for preventing keratin materials from becoming dull or dirty.

9. The use of ergothioneine and/or its derivatives in or for the preparation of an antipolluting composition, which is preferably a cosmetic composition, for topical application.

10. The use as claimed in claim 9, characterized in that said antipolluting composition contains from 0.005% to 10% and preferably from 0.01% to 5% by weight of ergothioneine and/or its derivatives relative to the total weight of the composition.

11. The use as claimed in either of claims 9 and 10, characterized in that said composition also contains at least one other antipollution compound.

12. The use as claimed in claim 11, characterized in that said antipollution compound is chosen from anthocyans and/or derivatives thereof, compounds containing a thioether, sulfoxide or sulfone function, heavy metal-chelating agents such as, for example, N,N′-dibenzylethylenediamine-N,N′-diacetic acid derivatives, antioxidants, ellagic acid or extracts of plants such as those of the Pontederiacea family.

13. The use as claimed in any one of claims 9 to 12, characterized in that the composition also contains a cosmetically acceptable medium consisting of water and/or at least one organic solvent chosen from the group consisting of hydrophilic organic solvents, lipophilic organic solvents and amphiphilic solvents, or mixtures thereof.

14. The use as claimed in claim 13, characterized in that the organic solvents are chosen from the group consisting of monofunctional or polyfunctional alcohols, optionally oxyethylenated polyethylene glycols, polypropylene glycol esters, sorbitol and its derivatives, dialkyl isosorbides, glycol ethers, polypropylene glycol ethers and fatty esters.

15. The use as claimed in claim 13 or 14, characterized in that the organic solvent(s) represent(s) from 5% to 98% relative to the total weight of the composition.

16. The use as claimed in any one of claims 11 to 15, characterized in that it also comprises at least one fatty phase.

17. The use as claimed in claim 16, characterized in that the fatty phase represents from 0 to 50% relative to the total weight of the composition.

18. The use as claimed in any one of claims 9 to 17, characterized in that it also contains at least one additive chosen from the group consisting of standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, dyestuffs, bactericides and antidandruff agents.

19. A composition as defined in any one of claims 9 to 18, characterized in that it is in the form of an aqueous, aqueous-alcoholic or oily solution, an oil-in-water or water-in-oil or multiple emulsion, an aqueous or oily gel, a liquid, pasty or solid anhydrous product, or a dispersion of oil in an aqueous phase using spherules.

20. The composition as defined in any one of claims 9 to 19, characterized in that the composition has the appearance of a white or colored cream, an ointment, a milk, a lotion, a serum, a paste, a mousse or solid.

21. The composition as defined in any one of claims 9 to 20, characterized in that it has a pH of between 3 and 8 and preferably between 5 and 7.

22. A cosmetic treatment process for protecting the body against the effects of pollution, characterized in that it consists in applying to keratin material a cosmetically effective amount of ergothioneine and/or its derivatives as defined in any one of claims 1 to 8.

23. A cosmetic treatment process for protecting the body against the effects of pollution, characterized in that it consists in applying to keratin material a cosmetic composition as defined in any one of claims 9 to 21.

24. (New) An antipollution composition for topical application for protecting skin and keratin material from harmful effects of pollution, comprising an antipollution effective amount of an antipollution agent which is ergothioneine and/or a derivative thereof.

25. (New) The composition of claim 24, wherein ergothioneine and/or the derivative thereof correspond to formula (I): 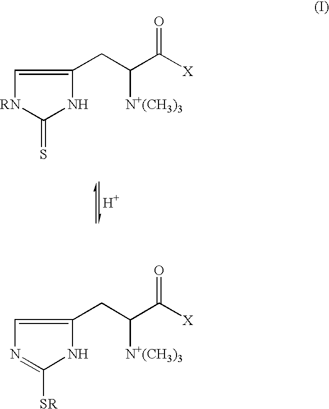
in which:
X represents a group —O—R′ or —NR′R″;
R, R′ and R″ represent, simultaneously or independently of each other, a hydrogen atom, a linear or branched alkyl, alkenyl or acyl group containing from 1 to 18 carbon atoms, optionally containing a hetero atom in the chain, optionally substituted with a hydroxyl group and optionally being esterified, a halogen atom, a carboxylic group or its derivative, an amine group or its derivative, or a salt thereof.

26. (New) The composition of claim 25, in which R, R′ and R″ denote, simultaneously or independently of each other, a hydrogen atom or an ethyl, acetyl or acyl group and X denotes NH₂.

27. (New) The composition of claim 25, wherein the compound of formula (I) is ergothioneine.

28. (New) The composition of claim 24, wherein the compound of formula (I) is L-(+)-ergothioneine.

29. (New) The composition of claim 24, wherein the antipollution agent is effective for protecting skin and keratin material against the effects of toxic gases.

30. (New) The composition of claim 29, wherein the antipollution agent is effective for protecting skin and keratin material against the effects of ozone.

31. (New) The composition of claim 24, wherein the antipollution agent is effective for improving cell respiration and/or for reducing desquamation and/or for preventing keratin material from becoming dull or dirty.

32. (New) The composition of claim 24, wherein the composition is a cosmetic composition.

33. (New) The composition of claim 32, wherein the composition contains from 0.005 to 10% by weight of ergothioneine and/or a derivative thereof relative to the total weight of the composition.

34. (New) The composition of claim 32, wherein the composition contains from 0.01 to 5% by weight of ergothioneine and/or a derivative thereof relative to the total weight of the composition.

35. (New) The composition of claim 32, wherein the composition further comprises at least one other antipollution agent.

36. (New) The composition of claim 35, wherein the other antipollution agent is an anthocyan and/or a derivative thereof, a compound containing a thioether, sulfoxide or sulfone function, a heavy metal-chelating agent, an antioxidant, ellagic acid or an extract of a plant.

37. (New) The composition of claim 36, wherein the the metal-chelating agent is a N,N′-dibenzylethylenediamine-N,N′-diacetic acid derivative.

38. (New) The composition of claim 36, wherein the plant is of the Pontederiacea family.

39. (New) The composition of claim 32, wherein the composition further comprises a cosmetically acceptable medium which is water and/or at least one organic solvent which is a hydrophilic organic solvent, a lipophilic organic solvent, an amphiphilic solvent, or mixtures thereof.

40. (New) The composition of claim 39, wherein the organic solvent is a monofunctional or polyfunctional alcohol, a polyethylene glycol which is optionally oxyethylenated, a polypropylene glycol ester, sorbitol or a derivative thereof, a dialkyl isosorbide, a glycol ether, a polypropylene glycol ether or a fatty ester.

41. (New) The composition of claim 39, wherein the organic solvent represents from 5 to 98% by weight relative to the total weight of the composition.

42. (New) The composition of claim 35, wherein the composition further comprises at least one fatty phase.

43. (New) The composition of claim 42, wherein the fatty phase represents from 0 to 50% by weight relative to the total weight of the composition.

44. (New) The composition of claim 32, wherein the composition further comprises at least one additive which is an aqueous or lipophilic gelling agent, a thickener, a hydrophilic or lipophilic active agent, a preserving agent, an antioxidant, a fragrance, an emulsifier, a moisturizer, a pigmenting agent, a depigmenting agent, a keratolytic agent, a vitamin, an emollient, a sequestering agent, a surfactant, a polymer, an acidifying or basifying agent, a filler, a free-radical scavenger, a ceramide, a sunscreen, an insect repellent, a slimming agent, a dyestuff, a bactericide or an antidandruff agent.

45. (New) The composition of claim 32, wherein the composition is an aqueous, aqueous-alcoholic or oily solution, an oil-in-water or water-in-oil or multiple emulsion, an aqueous or oily gel, a liquid, a pasty, a solid anhydrous product, or a dispersion of oil in an aqueous phase containing spherules.

46. (New) The composition of claim 32, wherein the composition is a white or colored cream, an ointment, a milk, a lotion, a serum, a paste, a mousse or a solid.

47. (New) The composition of claim 32, wherein the composition has a pH of between 3 and 8.

48. (New) The composition of claim 32, wherein the composition has a pH of between 5 and 7.

49. (New) A treatment process for protecting the body against the harmful effects of pollution, comprising applying to skin or keratin material the composition of claim 24.

50. (New) A cosmetic treatment process for protecting the body against the harmful effects of pollution, comprising applying to skin or keratin material the composition of claim 32.
